(19) United States
(12) Patent Application Publication
(10) Pub. No.: US 2011/0301502 A1
(43) Pub. Date: Dec. 8, 2011

(54) IN-VESSEL POSITIONING DEVICE

(76) Inventor: Sukhjit Gill, Oakbrook, IL (US)

(21) Appl. No.: 13/215,076

(22) Filed: Aug. 22, 2011

Related U.S. Application Data
(63) Continuation-in-part of application No. 13/027,102, filed on Feb. 14, 2011.
(60) Provisional application No. 61/304,265, filed on Feb. 12, 2010.

(51) Int. Cl. A61M 25/01

(52) U.S. Cl. 600/585

(57) ABSTRACT

A positioning device is disclosed. The device comprises a distal end, a proximal end, and an elongated body disposed in-between, wherein the elongated body is configured to accommodate at least a portion of the apparatus, and wherein a cross-sectional area of a portion the positioning device is smaller than a cross-sectional area of the apparatus and the cross-sectional area of a portion the positioning device is configured to be expandable.

### CROSS-REFERENCES TO RELATED APPLICATIONS

This application is a continuation-in-part of U.S. patent application Ser. No. 13/027,102, filed on Feb. 14, 2011, which claims the benefit and priority of U.S. Provisional Application No. 61/304,265, filed on Feb. 12, 2010, the full disclosure of the above referenced applications are incorporated herein by reference.

### FIELD OF THE INVENTION

This disclosure relates generally to devices, systems and methods for positioning an apparatus in a vessel.

### DESCRIPTION OF THE RELATED ART

Coronary artery disease is the most common cause of death in the adult population in both sexes in the United States of America. Chronic total occlusion (CTO) is the complete blockage of a blood vessel and usually has serious consequences, such as a heart attack, if not treated in a proper, safe and timely fashion. The cause of blockage could be the deposition of atheromatous plaque, old thrombus or similar other deposits in the body vessel. When these coronary lesions (CTOs) become very severe, they can either be treated by coronary intervention using catheters or coronary bypass surgery. A popular method of removal of such occlusions is by coronary intervention as it is less invasive than surgery.

Coronary intervention may include the following steps: A) coronary cannulation: A long catheter is advanced through the femoral artery near the groin area to the ostium of the coronary arteries carrying the lesions. Radiopaque contrast may be injected to identify the site of blockage in the lumens of the vasculature. B) Guidewire placement: A soft non-traumatic guidewire is advanced through the coronary catheter. This guidewire crosses the obstructive lesion and is advanced through the entire length of the vessel. This guidewire acts as a track over which further instrumentation is advanced. This step is often the pivotal point in determining the success of the intervention. The placement of the guidewire is the most challenging aspect in complicated cases for the physician. C) Stent deployment: After the wire is properly positioned, a catheter that carries a stent at its distal end over an inflatable part of this catheter is advanced over the guidewire. The stent is positioned accurately at the site of the blockage. After the stent has been properly positioned the balloon is inflated thus releasing the stent and at the same time flattening the plaque. D) Post procedure confirmation: The guidewire and catheter, which was carrying the stent, are removed and a post-procedure angiogram is done to demonstrate the success of the intervention.

Safe and successful recanalization of an occluded body vessel depends on precise positioning and insertion of the guidewire into the occluded body vessel. This involves steering the guidewire to negotiate curves and bifurcations of vessels. Some lesions are present in branches that diverge from the parent vessel at varying angles, as much as 90 degrees or more. In general, the guidewire must often pass through several bends and curves before reaching the target lesion and these obstacles need different angulation and curvature of the guidewire for successful passage. The situation becomes more challenging when occlusions are present in narrow branch body vessels that are to be accessed by advancing a soft, non-traumatic tip guidewire through a main body vessel leading to the branch vessel. In such cases, the chances of erroneous diversion of such a guidewire into an unintended location such as the subintimal space or puncturing the vessel wall are very high.

Known methods to access a targeted branch body vessel either use catheter devices that have deflectable tips or employ deflectable tip guidewires. These devices have a distal tip which is capable of being deflected by the operator to a desired angle for successful penetration into a targeted branch body vessel. Devices and methods for placing bifurcated stents at branch locations in a main body vessel also use mechanisms to deflect guidewires into side branches.

While using the deflectable tip catheters and guidewire devices to access a targeted branch body vessel, the device is inserted into a main body vessel to a suitable location near the bifurcation point and thereafter the deflectable tip of the device is used to steer a guidewire into the targeted branch body vessel.

U.S. Patent Publication No. 2009/0264980 to Mackay and U.S. Pat. No. 7,089,063 to Lesh et al. and U.S. Patent Publication No. 2009/0005755 to Keith et al. disclose catheters which employ deflectable tips for placing a guidewire into a targeted branched body vessel. As a variation, some catheters may have a flexible tip at the distal end which can be curved in any direction by the operator. For guiding a guidewire into a branched body vessel, these catheters are inserted in the main body vessel and the curved tip of the catheter is placed pointing towards target branch body vessel. Thereafter, a guidewire is advanced through the catheter and into target branch of the body vessel.

U.S. Pat. No. 5,916,194 to Jacobsen et al. discloses an apparatus for directing a guidewire from a catheter to a target branch vessel. This apparatus comprises a catheter with a guidewire lumen, a shapeable distal end of the catheter, an opening in the catheter's distal sidewalls and an expandable balloon at the distal end. The distal end of the catheter is deflectable by expanding the balloon to deflect and guide a guidewire into a target body vessel.

U.S. Patent Publication No. 2004/0116832 to Friedrich et al. describes a catheter arrangement for guiding a guidewire into a branch body vessel. The device comprises two catheters which are arranged coaxially and are movable with respect to each other. The inner catheter has a pre-bent distal tip for guiding a guidewire into a target body vessel. The tilt of the pre-bent distal tip of the inner catheter can be controlled by the axial movement of the inner catheter in the outer catheter.

U.S. Pat. No. 7,371,248 to Dapolito et al. discloses a guidewire and method for steering it through tortuous vessels. This type of guidewire comprises a hollow shaft and a core wire therein and a tubular protection element at its distal end. The shaft and the core wire control deployed and collapsed configurations of the tubular protection element. Further, the application of axial tension on the protection element creates a curvature at the distal end of the guidewire which is used for steering the guidewire through tortuous vessels.

U.S. Patent Publication No. 2006/0259009 to Murray, published PCT application WO/2006/046244A2 to Turgeman et al. and U.S. Patent Publication No. 2009/0306757 to Meyer et al. disclose apparatus for diverting a guidewire through a bifurcated passageway. The device of the Turgeman publication features an elongated hollow shaft bifurcated by a partitioning element at the distal section into separate first and second lumens, with suitable feature for deflecting a guidewire. U.S. Patent Publication No. 2009/0306757 to Meyer et al. disclosing a wiring assist device includes guidewire housing members and multiple lumen arrangement for parallel and angled orientation of the guidewires. U.S. Patent Publication No. 2006/0259009 to Murray discloses a guidewire loader catheter having two lumens attached tangentially to each other with one lumen extending beyond the other at the distal end.

The guidewire positioning devices described above may suffer from various complexities and/or constraints of operation, structure and size. Even when the operator succeeds in positioning the distal end of the guidewire into a targeted branched body vessel by any of the above stated methods, the operator may not be able to successfully and safely advance the guidewire further into the branched vessel due to problems of guidewire coiling up or slipping back of the guidewire into the main body vessel on repeated pushing. Additionally, due to the anatomy or composition of the vessel and/or an occlusion within the vessel, it may be difficult to position an apparatus at a desired location within the vessel. For example, it may be difficult to position a stent within an occluded vessel due to the torturous vasculature, anatomy of the occlusion cap, the hardness of the occlusion, or the like. The difficulty in positioning the apparatus may lead to repeated attempts and the application of additional force by the operator, which may cause complications such as damage to the vascular wall. Therefore, there exists the need for devices, methods, and systems where a positioning device is configured to position or facilitating the positioning of an apparatus in the vessel.

### SUMMARY

Described herein are devices, methods, and systems for positioning an apparatus in a vessel.

In one aspect, a device for positioning an apparatus in a vessel comprises a distal end, a proximal end, and an elongated body disposed in-between. The elongated body of the positioning device is configured to accommodate at least a portion of the apparatus. A cross-sectional area of a portion of the positioning device is configured to be smaller than a cross-sectional area of the apparatus and wherein at least portion of the cross-sectional area of the positioning device is configured to be expandable or to increase.

In another aspect, at least a portion of the elongated body of the positioning device is configured to decrease in cross-sectional area from the proximal to the distal end. In another aspect, the positioning device comprises a longitudinal opening disposed along a side of the elongated body. The longitudinal opening may be configured to allow the cross-sectional area of at least a portion of the elongated body to expand or to increase. In one aspect, the longitudinal opening may be defined by a first and second portion of the elongated body. In one aspect, the two portions are configured to overlap. In another aspect, the two portions are elastically separable. In one aspect, the two portions are configured to move away from each other when force is applied to expand the cross-sectional area of at least a portion of the elongated body.

In yet another aspect, the positioning device comprises a wire configured to support the elongated body. In one aspect, the wire may extend through at least a portion of a length of the elongated body. In another aspect, the positioning device further comprises a torquing means to transfer torque to the wire.

Also disclosed herein are methods of positioning an apparatus in a vessel. In one aspect, a method of positioning an apparatus in a vessel comprises inserting a positioning device comprising a distal end, a proximal end and an elongated body disposed in between into the vessel, inserting at least a portion of the apparatus into the elongated body of the positioning device, wherein a cross-sectional area of the positioning device is smaller than cross-sectional area of the apparatus, separating the apparatus and the positioning device by expanding or increasing the cross-sectional area of the distal end and advancing the apparatus through the positioning device, and positioning the apparatus in the vessel.

In one aspect, the positioning device comprises a longitudinal opening defined by a first portion and a second portion, wherein the separating is achieved by moving the first and second portion away from each other.

In another aspect, a portion of an occlusion in the vessel may be penetrated using the distal end of the positioning device and positioning the apparatus at the occlusion in the vessel.

In yet another aspect, the apparatus configured to be positioned by the positioning device is a stent and the stent is inserted into the positioning device in a collapsed configuration. The stent may be expanded once the stent has been positioned in the vessel.

This, and further aspects of the present embodiments are set forth herein.

### BRIEF DESCRIPTION OF THE DRAWINGS

FIG. 1 illustrates one embodiment of a positioning device comprising a longitudinal opening.

FIG. 2 illustrates one embodiment of a positioning device comprising a longitudinal opening and a diagonal opening at its proximal end.

FIGS. 3A-B illustrate cross-sectional views of two embodiments of the positioning device.

FIGS. 4A-B illustrate one embodiment of the positioning device and an apparatus.

FIG. 5 illustrates a flow diagram illustrating one embodiment of positioning a stent in a vessel using the positioning device.

FIGS. 6A-D illustrate steps of positioning a stent in a vessel using the positioning device according to one embodiment.

### DETAILED DESCRIPTION

Although the detailed description contains many specifics, these should not be construed as limiting the scope of the disclosure but merely as illustrating different examples and aspects of the disclosure. It should be appreciated that the scope of the disclosure includes other embodiments not discussed in herein. Various other modifications, changes and variations which will be apparent to those skilled in the art may be made in the arrangement, operation and details of the method and apparatus disclosed herein without departing from the spirit and scope of the disclosure as described here.

The present devices, methods, and systems contemplate embodiments where a positioning device is configured to position or to facilitate the positioning of an apparatus in the vessel. Due to the anatomy or composition of the vessel and/or an occlusion within the vessel, it may be difficult to position an apparatus at a desired location within the vessel. For example, it may be difficult to position a stent within an occluded vessel due to the torturous vasculature, anatomy of the occlusion cap, the hardness of the occlusion, or the like. The difficulty in positioning the apparatus may lead to repeated attempts and the application of additional force by the operator, which may cause complications such as damage to the vascular wall.

Briefly stated, the devices, methods, and systems disclosed herein contemplate embodiments where a positioning device is configured to position an apparatus, such as a stent, in the vessel by housing the apparatus within an elongated body of the positioning device. Once the apparatus is positioned at or near a desired location, the positioning device is configured to separate from the apparatus such that the apparatus is positioned in the vessel. The disclosed embodiments may minimize repeated attempts of positioning an apparatus into a vessel and enable rapid and safe apparatus positioning procedures to be carried out, while increasing patient comfort and safety as well as reducing fatigue to the operator.

As referred to herein, a vessel could be any vessel or artery in which blood flows through the hollow tubular cavity as well as any duct within the body. Also, referred to herein, an apparatus may be any device used to treat an indication in the vessel, such as weakened vessel, occlusion in the vessel, or the like. In one embodiment, the apparatus may be a device configured to penetrate, weaken, and/or recanalize an occlusion such as an atheromatous plaque, an old thrombus, or other similar deposit. In another embodiment, the apparatus may be a stent, such as a drug eluting stent, a covered stent, balloon expandable stent, bare-metal stent, self-expanding stent, or the like that may be inserted into the vessel to support the vessel wall and/or to prevent restenosis. Additionally and optionally, the apparatus may be a visualization element, therapeutic agent delivery element, or the like.

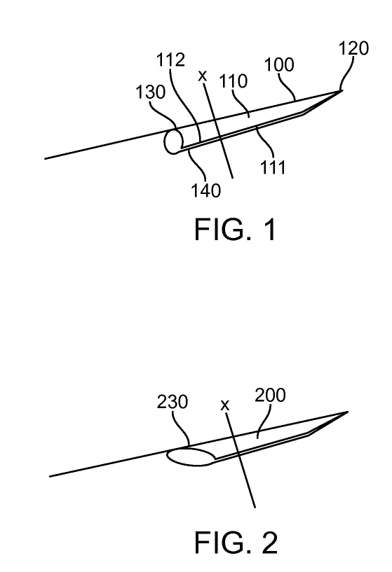

As shown in FIG. 1, the positioning device 100 comprises a distal end 120, a proximal end 130 and an elongated body 110 disposed in-between. In one embodiment, the elongated body 110 comprises a first portion 111 and a second portion 112, wherein the first and the second portions 111 and 112 define a substantially longitudinal opening 140 that extends along a length of the elongated body 110, such as the entire length of the elongated body 110.

Alternatively, in another embodiment the elongated body disposed in-between the proximal end and the distal end may be substantially continuous without any longitudinal openings. In yet another embodiment, the elongated body may comprise one or more openings that extend along a partial length of the elongated body. For example, the elongated body may comprise one or more slit openings disposed longitudinally on the elongated body, wherein the slit openings extend along a portion of the elongated body instead of the entire length of the elongated body as shown in FIG. 1. Alternatively, the slit openings may extend diagonally, or the slit openings may assume a predetermined shape or pattern such as a zigzag pattern or any other pattern. In yet another embodiment, the slit openings may be disposed cross-wise along the elongated body, wherein the slit openings extend along a partial circumference of the elongated body. It is further contemplated that the elongated body may comprise one or more opening configurations, for example, in one embodiment the elongated body may comprise a longitudinal opening that extends along the entire length of the elongated body and one or more slit openings that extend along a partial length of the elongated body.

The cross-sectional area or the diameter of positioning device 100 may be configured to decrease from a proximal end 130 towards the distal end 120, whereby the positioning device 100 assumes a substantially tapered or conical configuration. The tapered configuration improves maneuverability and navigation of the positioning device 100, particularly in narrow or tortuous vasculature. Furthermore, the tapered configuration where the distal end 120 is substantially narrow may create a sharp tip that facilitates occlusion penetration. Alternatively, the guidewire positioning device may comprise a substantially cylindrical or blunt distal end.

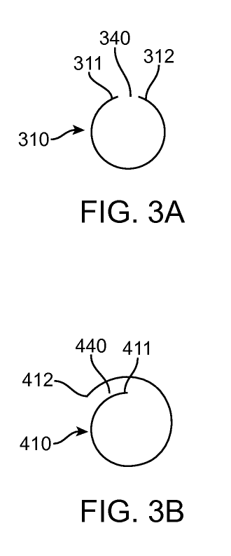

In one embodiment, the positioning device 100 may comprise a substantially flat opening at its proximal end 130 as shown in FIG. 1. Alternatively, as shown in FIG. 2, an embodiment of the positioning device 100 may comprise a substantially diagonal opening at the proximal end 130. The substantially diagonal opening may allow more maneuverability and may facilitate insertion and/or removal of the apparatus.

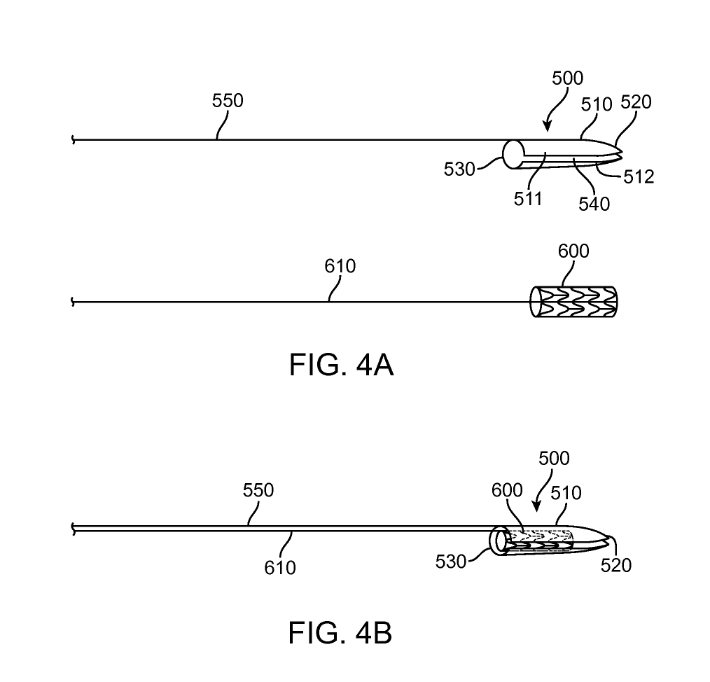

Two cross-sectional views of the positioning device are shown in FIGS. 3A and 3B. In one embodiment, as seen in FIG. 3A, the longitudinal opening 340 is defined by a first portion 311 and second portion 312 of the elongated body 310, wherein the two portions are non-overlapping. In such embodiment, the longitudinal opening 340 may be in the form of a gap on the side wall of the elongated body 310. In another embodiment, as seen in FIG. 3B, the longitudinal opening 440 is defined by a first portion 411 and second portion 412, wherein the two portions are overlapping. In such embodiment, the cross-section of at least a portion of the positioning device may assume a spiral, or folded form as seen in FIG. 3B.

Additionally and optionally, the positioning device may be constructed of a flexible, elastic and/or shape-memory material. For example, the positioning device may be constructed of various materials that exhibit sufficient strength, elasticity, and/or flexibility such as polyvinyl chloride, cross-linked polyethylene, polyethylene terephthalate (Dacron), PET, nylon, latex, silicone, or the like. Additionally, the positioning device may be coated for lubrication, for abrasion resistance, or to deliver an anti-coagulatory drug or other therapeutic agents.

In one embodiment, the first and/or the second portions of the elongated body may be configured to alter the size and/or shape of the longitudinal opening and thereby altering the size, area, volume, and/or shape of the elongated body when a force is applied to the elongated body. In another embodiment, where the elongated body comprises one or more slit openings, the size and/or shape of the slit openings may be altered and thereby altering the size, area, volume, and/or shape of the elongated body. In yet another embodiment, where the elongated body is substantially continuous without any longitudinal openings, the size, area, volume and/or shape of the elongated body may be altered due to the elastic nature of the elongated body.

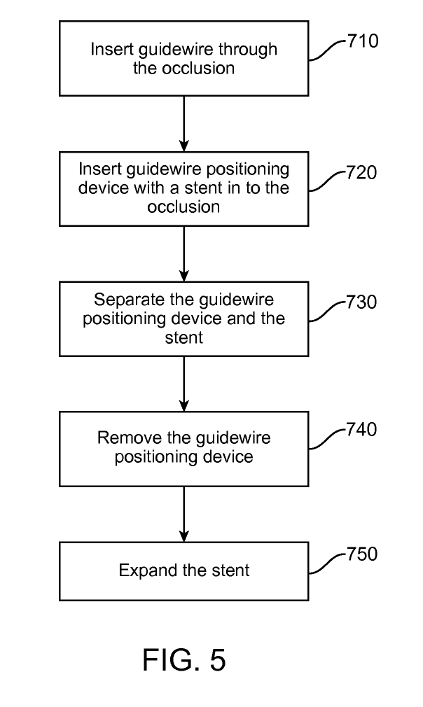

Referring now to FIGS. 4A-B, where one embodiment of the positioning device is shown along with one exemplary apparatus. As seen FIG. 4A, the positioning device 500 comprises a distal end 520, a proximal end 530 and an elongated body 510 disposed in-between. The first portion 511 and the second portion 512 of the elongated body 510 define a longitudinal opening 540. The apparatus as seen in FIG. 4A, is exemplarily shown as a stent 600, such as a drug eluting stent, a covered stent, balloon expandable stent, bare-metal stent, self expanding stent, or the like. The stent is shown in its collapsed form placed over a stent catheter 610. The stent catheter 610 may further comprise an inflatable balloon (not shown) disposed on the distal end under the stent 600 that is configured to expand the stent upon inflation of the balloon.

Additionally and optionally, the positioning device 500 may further comprise a support wire 550 configured to provide support for the positioning device 500 and to allow an operator to maneuver the positioning device 500. In one embodiment, the support wire 550 extends through substantially an entire length of the positioning device 500, thereby directly supporting substantially the entire length of the positioning device 500; in another embodiment, the support wire 550 extends through a portion of the positioning device 500.

The support wire 550 may comprise a single wire or multiple wires each having a solid cross-section. The support wire 550 may be constructed of helically wound wire or wires. Additionally, the support wire 550 may be either hollow or solid in one or more portions to maintain optimum flexibility during operation. In one embodiment, a portion of the support wire 550 may be configured with a decreasing diameter from the proximal end to the distal end. In another embodiment the stiffness of a portion of the wire shaft may be configured to decrease from the proximal end to the distal end. The support wire 550 may be constructed of any material with suitable properties that are well known in the art for surgical applications such as stainless steel, cobalt alloy, nickel-titanium, and the like.

The support wire 550 may be configured to be torquable by a torquing means (not shown) disposed on or near the proximal end of the support wire 550 for providing rotational torque to the support wire 550. Rotational torquing may help to position the positioning device 500 at a desired orientation or location in the vessel. The torquing means may be in the form of a spindle, a steering disk, a wheel, or the like that are known in the art. Furthermore, the torquing means may be configured to distinguish between a support wire 550, a guidewire, stent catheter 650, or the like by tactile feedback to facilitate the operation under decreased or minimal illumination.

The elongated body 510 of the positioning device 500 is configured to accommodate at least a portion of the stent 600. As seen in FIG. 4B, the elongated body 610 may be configured to accommodate the entire length of the collapsed stent 600. It is further contemplated that the elongated body 510 may be configured to accommodate a portion of the stent 600 such that a portion of the stent 600 is disposed within the elongated body 510 and a portion of the stent 600 is disposed outside of the elongated body 510. Additionally, in one embodiment, a cross-sectional area of the distal end 520 of the positioning device 500 is configured to be smaller than a cross-sectional area of the stent 600. In such embodiment, when the stent 600 is housed within the positioning device 500, the stent 600 is prevented from exiting the positioning device 500 through the distal end 520.

The cross-sectional area of at least a portion of the positioning device 500 is configured to be expandable. In one embodiment, the first and/or second portion of the elongated body is configured to be moveable such that when the first portion and the second portion are moved away from each other, the cross-sectional area of a portion of the positioning device 500 is increased. The movement of the first and/or second portion of the positioning device 500 may be caused by a force applied to the elongated body 510. In one embodiment, a force is applied to an internal surface of the elongated body 510 thereby forcing the first and/or the second portion to move away from each other.

Specifically, in one embodiment, a force may be applied to the first and/or second portion by means of the apparatus such as the stent 600. In such embodiment, the force may be applied in the distal direction towards the distal end 520 of the positioning device 500 by the operator, wherein the force is transmitted to the stent 600 through the stent catheter 610. The distal directional force causes a portion of the stent 600 with a larger cross-sectional area than a portion of the positioning device 500 to transmit the force to a portion of the elongated body 510, such as the first and/or second portion. The force transmitted to the elongated body 510 thereby moves or pulls the first and/or second portion away from each other, which results in an increase to the cross-sectional area of a portion of the positioning device 500. In one embodiment, the cross-sectional area of the distal end 520 of the positioning device 500 is configured to increase upon receiving the distal directional force such that the cross-sectional area of the distal end 520 is increased by a degree where that the stent 600 may exit through the distal end 520 of the positioning device 500. Alternatively, in one embodiment where the elongated body of the positioning device is substantially continuous such that elongated body 510 is devoid of the longitudinal opening, the cross-sectional area of the elongated body may be increased as a result of the distal directional force as a result of the elastic and/or expandable property of the elongated body.

Additionally and optionally, the longitudinal opening 540 may be configured to accommodate at least a portion of the apparatus and/or a device associated with the apparatus such that a portion of the apparatus and/or the associated device may separate from the positioning device by passing through the longitudinal opening 540. In one embodiment, the longitudinal opening 540 may be configured to accommodate the stent catheter 610. In such embodiment, the stent catheter 540 may be inserted or removed from the positioning device 500 through the longitudinal opening 540.

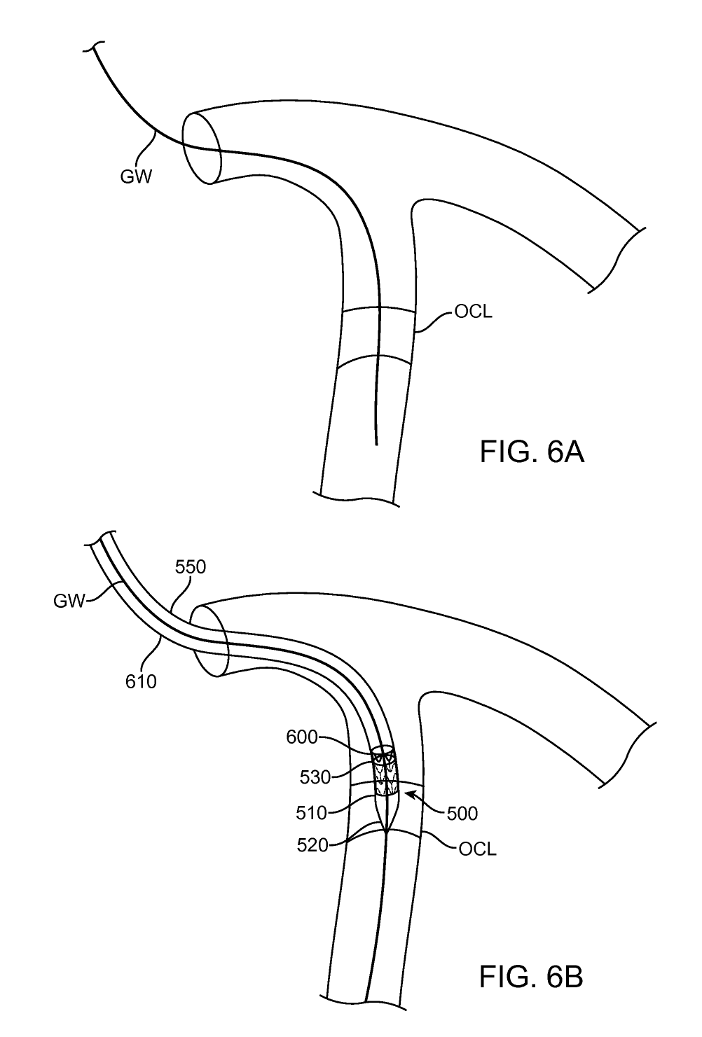

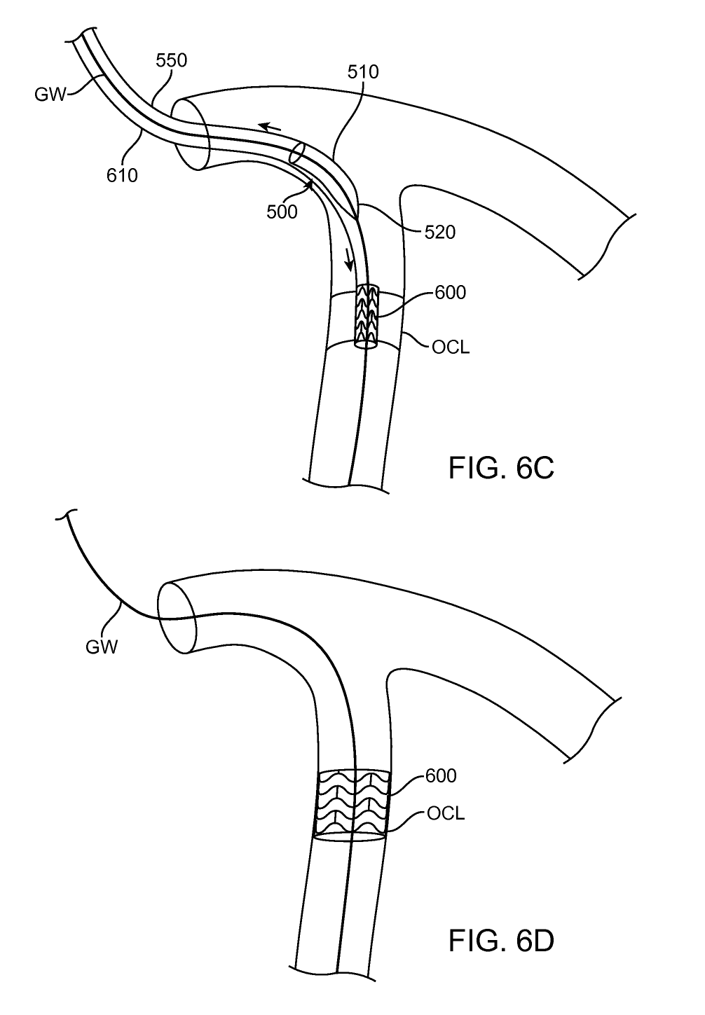

Referring now to FIG. 5, which illustrates a flow diagram of one method of positioning an apparatus exemplarily referred to as a stent according to the present embodiments and with reference to FIGS. 6A-D. At step 710 and as shown in FIG. 6A, a guidewire GW is inserted into the vessel and through an occlusion OCL. Various methods of inserting and guiding a guidewire into a vessel such as a main body vessel or a branch vessel may be used. For example, it is contemplated that the guidewire GW may be guided into the vessel by using a guidewire positioning system as described in the co-pending U.S. patent application Ser. No. 13/027,102, which is herein incorporated by reference in its entirety. It is contemplated that various kinds of guidewires may be used. For example, the guidewire may be a stiff guidewire, an ultrasound guidewire and/or guidewires with active means for eliminating a portion of the occlusion such as RF devices, laser devices and the like. It is contemplated that the guidewire GW may be configured as a standard length wire used in cardiac procedures, such as a wire of approximately 180 cm. Alternatively, it is contemplated that the guidewire may be configured as any length or size appropriate for the operation. Additionally and optionally, it is contemplated that other surgical devices for treatment of vascular occlusions such as mechanical burrs may be inserted into the vessel to treat the occlusion prior to, contemporary to, or after the guidewire GW has be is inserted through the occlusion OCL.

At step 720 and as shown in FIG. 6B, the positioning device 500 is inserted into the vessel along the guidewire GW, wherein at least a portion of the positioning device 500 is inserted into the occlusion OCL. The distal end 520 of the positioning device may be configured as a tapered distal tip to facilitate the penetration of the occlusion OCL. As seen in FIG. 6B, a portion of the stent 600 is housed within the elongated body 510 of the positioning device 500. In one embodiment, the positioning device 500 may be inserted into the vessel prior to the stent 600. In such embodiment, the positioning device 500 may be inserted in the vessel along the guidewire GW, once at least a portion of the positioning device 500 is inserted into the occlusion OCL, the stent 600 is then inserted into the vessel along the guidewire GW where a portion of the stent 600 is received by the proximal end 530 of the positioning device 500 and a portion of the stent 600 is housed within the elongated body 510 of the positioning device 500.

Alternatively, in another embodiment the positioning device 500 and the stent 600 may be coupled prior to the insertion of the positioning device 500 and the coupled positioning device 500 and the stent 600 may be inserted in tandem along the guidewire GW into the vessel and a portion of the occlusion OCL.

At step 730 and as shown in FIG. 6C, once the positioning device 500 is inserted into the occlusion OCL and a portion of the stent 600 is housed within the elongated body 510 of the positioning device 500, the positioning device 500 is separated from the stent 600. In one embodiment, the separation is initiated by applying a force to an internal surface of elongated body 510 which causes the cross-sectional area of a portion of the positioning device 500 to increase such that the stent 600 may exit through the distal end 520 of the positioning device 500. As described above, the force may be applied to the first and/or second portion of the elongated body 510 by means of the stent 600, where the force may be applied in the distal direction towards the distal end 520 of the positioning device 500 by the operator. The distal directional force causes a portion of the stent 600 with a larger cross-sectional area than a portion of the positioning device 500 to transmit the force to a portion of the elongated body 510, such as the first and/or second portion. The force transmitted to the elongated body 510 thereby moves or pulls the first and/or second portion away from each other, which results in an increase to the cross-sectional area of a portion of the positioning device 500.

Thereafter, the stent 600 is pushed distally through the distal end 520 of the positioning device 500 such that the stent 600 traverses the positioning device 500. Additionally and optionally, In an embodiment where the positioning device 500 comprises a longitudinal opening as seen in FIG. 1, the stent catheter 610 may be separated from the positioning device 500 by manipulating the positioning device 500 and/or the stent catheter 610 such that the stent catheter 610 passes through the longitudinal opening of the positioning device. In one embodiment, the separation may be accomplished by moving the stent catheter 610, the positioning device 500 or both such that the stent catheter 610 is moved through the longitudinal opening. Thereafter, the positioning device 500 may be removed from the vessel by traversing in the proximal direction along the guidewire GW. Alternatively, in an embodiment where the positioning device 500 does not comprise a longitudinal opening, the positioning device 500 is moved proximally such that the positioning device 500 traverses along both the stent catheter 610 and the guidewire GW. Additionally and optionally, the stent 600 may be moved in the distal direction where the stent 600 further advances into the occlusion OCL.

At step 740, the stent 600 is transformed from the collapsed configuration into an expanded configuration whereby the expanded stent 600 occupies a greater volume within the vessel. The transformation of the stent 600 may be accomplished through various means depending on the nature of the stent 600. In one embodiment, where the stent 600 is a balloon expandable stent, a balloon (not shown) underneath the stent 600 is inflated, thereby expanding the stent 600.

It is contemplated that the various embodiments of the positioning device as described above may be constructed partially or completely of elastic material. Furthermore, the longitudinal or the slit openings may be configured to return to its original size or shape when the applied force is released. Furthermore, it is contemplated that the elongated body including the first and/or the second portions of the various embodiments may be configured to alter and to retain the altered size and/or shape for a period of time.

It is also contemplated that the various embodiments of the elongated body of the positioning device may be substantially cylindrical, or may assume various curvatures to facilitate insertion into the vessel. The curvature or tilt angle of the elongated body of the positioning device may be adjustable. In one embodiment, the curvatures of the elongated body may assume a substantially flat configuration to allow advancement through a vessel. Thereafter, the curvature of the elongated body may be adjusted such that the distal end of the positioning device may assume a curved configuration wherein the distal end may be placed at or near the branch vessel. It is further contemplated that the curvature may be adjusted throughout the operation to facilitate access and/or navigation within the body region.

The curvature or tilt angle of the elongated body may be pre-configured by using a shape memory material or other means known in the art during manufacture, or it may be configured by the operator prior to, or during the operation. It is further contemplated that the curvature may be variable and/or adjusted by the operator. In one embodiment, the angular orientation or curvature of the elongated body may be flattened by inserting a relatively stiffer portion of a support wire into the elongated body of the positioning device. The angular orientation may be varied as desired by withdrawing or advancing the softer portion of the support wire through the elongated body. Additionally and optionally, the elongated body may be embedded with a deformable wire, such as a plastically deformable wire, to configure the desired curvature or tilt angle.

It is further contemplated that the various embodiments of the positioning device may comprise radiopaque markers disposed on or near the proximal end and/or the distal end of the positioning device. The radiopaque markers may facilitate tracking the location of the positioning device, particularly, while the positioning device navigates through narrow and/or tortuous vasculature during the operation.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A device for positioning an apparatus in a vessel, comprising:
a distal end, a proximal end, and an elongated body disposed in-between;
wherein the elongated body is configured to accommodate at least a portion of the apparatus, and wherein a cross-sectional area of a portion of the positioning device is smaller than a cross-sectional area of a portion of the apparatus and the cross-sectional area of the elongated body is configured to be expandable.

2. The device of claim 1, wherein at least a portion of the elongated body decreases in cross-sectional area from the proximal to the distal end.

3. The device of claim 1, further comprising a longitudinal opening along a side of the elongated body.

4. The device of claim 3, wherein the longitudinal opening is defined by a first and second portion of the elongated body.

5. The device of claim 4, wherein the two portions are configured to overlap.

6. The device of claim 4, wherein the portions are elastically separable.

7. The device of claim 4, wherein the longitudinal opening is configured to allow the cross-sectional area of at least a portion of the elongated body to expand.

8. The device of claim 7, wherein the first and second portions are configured to move away from each other when a force is applied to expand the cross-sectional area of at least a portion of the elongated body.

9. The device of claim 1, further comprising a wire configured to support the elongated body.

10. The device of claim 9, wherein the wire extends at least a portion of a length of the elongated body.

11. The device of claim 9, further comprising a torquing means to transfer torque to the wire.

12. The device of claim 1, wherein the apparatus is a stent.

13. A method of positioning an apparatus in a vessel, comprising:
inserting a positioning device comprising a distal end, a proximal end and an elongated body disposed in-between into the vessel;
inserting at least a portion of the apparatus into the elongated body of the positioning device, wherein a cross-sectional area of the positioning device is smaller than a cross-sectional area of the apparatus;
separating the apparatus and the positioning device by expanding the cross-sectional area of the positioning device and advancing the apparatus through the positioning device; and
positioning the apparatus in the vessel.

14. The method of claim 13, wherein the positioning device comprises a longitudinal opening defined by a first portion and a second portion, wherein the separating is achieved by moving the first and second portions away from each other.

15. The method of claim 13, further comprising penetrating a portion of an occlusion in the vessel using the distal end of the positioning device and positioning the apparatus at the occlusion in the vessel.

16. The method of claim 14, wherein the apparatus is a stent and the stent is inserted into the positioning device in a collapsed configuration.

17. A method of positioning an apparatus in a vessel, comprising:
inserting a positioning device comprising a distal end, a proximal end and an elongated body disposed in-between into the vessel;
wherein the positioning device houses an apparatus such that at least a portion of the apparatus is disposed within the elongated body, wherein a cross-sectional area of the positioning device is smaller than a cross-sectional area of the apparatus;
separating the apparatus and the positioning device by expanding the cross-sectional area of a portion of the positioning device advancing the apparatus through the positioning device; and
positioning the apparatus in the vessel.

18. The method of claim 18, wherein the positioning device comprises a longitudinal opening defined by a first portion and a second portion, wherein the separating is achieved by moving the first and second portions away from each other.

19. The method of claim 18, further comprising penetrating a portion of an occlusion in the vessel using the distal end of the positioning device and positioning the apparatus at the occlusion in the vessel.

20. The method of claim 18, wherein the apparatus is a stent and the stent is inserted into the positioning device in a collapsed configuration.
